(19) United States
(12) Patent Application Publication
(10) Pub. No.: US 2010/0313632 A1
(43) Pub. Date: Dec. 16, 2010

(54) Method For Removing A Coating Comprising A Hazardous Compound From A Surface

(75) Inventor: Johann Ritt, Wegscheid (DE)

Correspondence Address: Tolpin & Partners, PC, 11 S. LaSalle Street, Suite 2900, Chicago, IL, 60603, US

(73) Assignee: SK-Patent, Munchen (DE)

(21) Appl. No.: 12/839,544

(22) Filed: Jul. 20, 2010

(30) Foreign Application Priority Data
Aug. 26, 2008 (DE) EP 080105014

(51) Int. Cl. G01N 33/00; B32B 43/00

(52) U.S. Cl. 73/28.01; 427/140

(57) ABSTRACT

The invention concerns a method for removing resp. renovating a layer resp. coating (4) from a surface (2) covered with the coating (4), wherein the coating (4) to be removed comprises a hazardous material. According to the method, a working step, which produces dust, such as in particular a grinding or sanding, is performed only on, resp. in a portion of, the surface, which has been coated with at least one newly applied priming coat layer (26). In a first aspect, the method comprises: (i) pre-treating the coating (4) to be removed resp. renovated, so that at least a portion of the coating is transformed into a moist resp. viscous state, (ii) removing portions (10) of the coating (4), which have been affected by the pre-treatment of step (i) at least in sub-areas of the portions, where the pre-treatment of step (i) has been applied, so that exposed parts of the surface are formed at least in these sub-areas, and (iii) applying, without performing an intermediate grinding beforehand, at least one priming coat layer (26) at least in sub-areas of the exposed parts (22) of the surface formed in step (ii). In a second aspect, a working step, which produces dust, such as in particular a grinding or sanding, is performed only on, resp. in a portion of the surface, which has been coated with at least one newly applied priming coat layer (26). In a third aspect, at least a portion of the coating (4) comprising the hazardous material is transformed into a moist resp. viscous state and is relieved resp. removed from the surface (2) without generating dust and/or flour. In a fourth aspect, remainders (20) of the coating (4) to be removed resp. renovated, which remain, after a step of relieving resp. removing loose resp. viscous portions of the coating from the surface (2) without generating dust, in recesses, e.g. holes and pores, extending deeper into the otherwise substantially exposed surface (22), are covered resp. masked. In a fifth aspect, remainders of the coating (4) to be removed resp. renovated, which remain attached to the surface (2) after a step of relieving resp. removing loose resp. viscous portions of the coating from the surface without generating dust, are covered resp. masked, e.g. by applying an impregnating agent (24) and/or a priming coat layer (26) onto the otherwise substantially exposed portions of the surface (2).

### BACKGROUND OF THE INVENTION

The present invention relates to a method for removing a layer resp. coating, for example a colour coat or a lacquer coat, which may in particular comprise a hazardous material, from a surface. For activities involving resp. for usages of materials comprising a hazardous material, there exist as a general rule regulations on the limitation of the liberation of these hazardous materials and/or regulations for the protection of the persons charged with the activities. The invention relates further to a method for verification of the conformity with these regulations upon removing a coating resp. layer comprising a hazardous material, from a surface.

Lacquer coats resp. colour coats, which may be applied e.g. on metal or wood surfaces and which must be removed resp. renovated following a weathering resp. decomposition or other wearing, are examples of coatings to be removed from surfaces. Until in the 1980's, paints and lacquers often comprised heavy metals, such as lead, or anorganic heavy metal compounds or heavy metal preparations, which have later been recognized and classified as hazardous materials. For activities on an object comprising a hazardous material resp. for activities involving a substance comprising a hazardous material, regulations are in force in many countries, including e.g. Germany and the United States on one hand side relating to the limitation of the liberation of the hazardous material into the environment and on the other hand side regulations for the protection of persons, who are in charge of the activities on the object resp. the activity involving the substance comprising a hazardous material. Such regulations may comprise protection measures resp. precautions, which are logistically resp. technically elaborate and expensive. In particular cases, one could refrain from the compliance with the prescribed protection measures resp. precautions, namely when the manner of performing the activities is designed, even without the prescribed protection measures resp. precautions such that a charge resp. contamination of the environment and of the persons charged with the activities with the hazardous material is harmless resp. not occurring, e.g. within prescribed maximum permissible limits.

For example, according to a summary of maximum permissible for the concentration of lead edited by GESTIS (German: Gefahrstoffinformationssystem der Deutschen Gesetzlichen Unfallversicherung; hazardous material information system of the German legally prescribed insurance against accidents), refer to http://www.dguy.de/ifa/de/gestis/stoffdb, a maximum limiting value of the concentration of lead permissible in air, expressed in milligrams of inhalable aerosols per cubic meter of air [mg/m3], is prescribed e.g. in Italy and Belgium at 0.15 over a period of 8 hours, in Germany and Japan at 0.1 over a period of 8 hours, in Austria at 0.1 over a period of 8 hours and at 0.4 as an instantaneously measured value. In the United States, the maximum permissible concentration of lead in air is imposed at less than 0.1 according to NIOSH (lead concentration in air, prescribed such that the lead concentration in the blood of a worker is less than 0.060 mg lead per 100 gram of blood, which corresponds to approx. 600 μg/l), and the maximum permissible concentration of dust, which contains lead, in air is imposed at 0.05 mg/m3 according to OSHA. In Germany, a maximum permissible limit of 400 μg/l for the concentration of lead in blood is prescribed, which is well below that of NIOSH in the United States.

For example, in the United States, the legislator has recognized that common renovation activities like sanding, cutting, and demolition can create hazardous lead dust and chips by disturbing lead-based paint, which can be harmful to adults and children. Accordingly, on Apr. 22, 2008, the Environmental Protection Agency (EPA) issued a rule requiring the use of lead-safe practices and other actions aimed at preventing lead poisoning. From December 2008, the rule has required that contractors performing renovation, repair and painting projects that disturb lead-based paint provide to owners and occupants of child care facilities and to parents and guardians of children under age six that attend child care facilities built prior to 1978 the lead hazard information pamphlet “Renovate Right: Important Lead Hazard Information for Families, Child Care Providers, and Schools”.

As from 22 Apr. 2010, contractors performing renovation, repair and painting projects that disturb lead-based paint in homes, child care facilities, and schools built before 1978 must be certified and must follow specific work practices to prevent lead contamination. All contractors should follow the following three simple procedures: (1) contain the work area, (2) minimize dust, and (3) clean up thoroughly. The new rule affects paid renovators who work in pre-1978 housing and child-occupied facilities, including: renovation contractors, maintenance workers in multi-family housing, and painters as well as other specialty trades.

Under the rule, child-occupied facilities are defined as residential, public or commercial buildings where children under age six are present on a regular basis. The requirements apply to renovation, repair or painting activities. The rule does not apply to minor maintenance or repair activities where less than six square feet of lead-based paint is disturbed in a room or where less then 20 square feet of lead-based paint is disturbed on the exterior. Window replacement is not minor maintenance or repair.

For example, in Germany, the TRGS (German: technische Regel für Gefahrstoffe, technical regulation for hazardous materials) no. 505 applies for activities on resp. involving substances comprising hazardous materials including a heavy metal, in particular lead. The TRGS 505 body of rules and regulations addresses employers and comprises specific protection measures for activities involving lead and anorganic lead compounds as well as compositions containing lead. Activities to which the TRGS 505 is applicable comprise the removing of lead-containing coatings, e.g. by burning off, by using abrasive methods (e.g. brushing, grinding and/or blasting) or by chemically stripping and/or by machining of lead, lead alloys or lead-containing lacquer coats by involving mechanical methods (grinding, polishing, chipping resp. chip removal) or thermal methods. Therefore, the removal of lacquer coats resp. colour coats of lead-containing paints, such as they have often been used until in the 1980's, also falls under this regulation. For the above mentioned activities, the TRGS 505 prescribes for example the following: sources of dust resp. powder must be encapsulated; working sites charged with lead must be connected to a stationary suction unit; mobile suction units must be provided in the working areas; the supply of uncharged fresh air resp. recirculation air must be secured by ventilation practice measures; pre-, intermediate and final products containing lead or lead-containing substances must be transported emission-free; floor surfaces in the working areas must be moistened in order to minimize diffuse emissions of (lead-containing) dust; working areas must be cleaned moistly at least daily; and specific measures for personal hygiene such as the cleaning of shoes with suitable equipment before leaving lead-charged working areas, changing of working clothes at least daily or also multiple times per shift and cleaning of working clothes before intermissions and at the end of a shift.

The encapsulation of sources of dust and the use of a suction unit can become technically resp. logistically complex and expensive in particular when renovating windows and window sashes in-situ at buildings, because a window to be renovated would have to be encapsulated on the exterior side and the inner side of the building and a suction unit would have to be provided within the encapsulated working area.

The compliance with the regulations of the TRGS 505 and supplementary regulations from commercial inspectorates is important in particular, when dust containing a hazardous material is generated in the of the work activities. This is the case with the presently common methods for removing coatings of paint resp. lacquer, because these methods are methods performed dry, i.e. in a dry environment, and comprise at least one abrasive working step, such as grinding or scraping. The generation of dust is particularly intensive when grinding is performed. When scraping is performed, bigger particles are generated in the beginning, but then also crumbs and thereby also dust.

The presently conventionally used methods for removing coatings of paint and/or lacquer comprising a hazardous compound, from wood, which are normally performed in-situ, comprise thermal methods wherein a stream of hot air, which has e.g. been generated using a hot air blower and has such an elevated temperature, that the wood just does not start burning, but the paint becomes soft and eventually brings up bubbles, and then the layer is removed abrasively, e.g. by scraping. Thereby, gases which are dangerous for the health of persons are generated due to the heat, and when scraping the layer, crumbs are generated, and when crumbling these, dust is generated. Alternatively, a gas torch can be used for heating the paint, in particular when the paint coating to be removed is present on a metal surface. Alternatively to the thermal equipment, the lacquer resp. paint coating to be removed can be irradiated using an UV (ultraviolet) lamp and subsequently be removed abrasively, whereby again crumbs and dust are generated.

Another category of well-established methods for removing paint resp. lacquer coating (depainting methods), which are performed in-situ, comprises mechanically removing the lacquer resp. paint coating, in particular by lacquer planeing, grinding or high pressure sanding. These methods comprise as a basic working step a dry, abrasive treating of the lacquer resp. paint coating, whereby dust containing the material of the layer to be removed is generated. The generation of dust is particularly strong when grinding is performed, which can in particular be facilitated using a mechanical tool, e.g. using an angle grinding machine.

GB 644 791 concerns the removing of layer compositions from coated substrates by a treatment using a paste produced from an aqueous solution of an alkali hydroxide of finely grinded lignin resulting from the hydrolysis of wood and an organic solvent. In an example, a layer consisting of such a paste and having a thickness of 1-2 mm is applied, using a timber plank, to a front of a building having applied thereon a layer of oil paint having a thickness of approximately 1 mm After a waiting period of 20 hours, the paste has dried and is broken up respectively open in scales. The scales are removed using a scraping knife together with a major portion of the paint layer composition. The remainder of the paint layer composition is removed by water using a hard brush. Thereafter, the surface is neutralised using a dissolved mineral acid. Thereafter, the front of the building is newly painted.

U.S. Pat. No. 3,391,084 discloses an organic paint stripping composition, which is non-inflammable, can be washed away using water and is of the solvent type, for stripping dry paint coatings from all kinds of metallic and non-metallic substrates. In an example, the composition is applied using a roller or by spraying or brushing method onto a surface provided with a paint coating. As a result, the coating forms bubbles of gas between the coating and the substrate, is softened and then rinsed away using water. On the substrate, there does not remain any coating or remainder of the coating, but a passivated layer. A new coat of paint is applied thereon, whereby no treatment of the surface is required.

U.S. Pat. No. 5,964,961 discloses a method for safely and economically removing lead-based paint coating from a substrate, the method comprising: (a) applying an aqueous solution onto the paint coating on the substrate; (b) drying the applied solution; (c) repeatedly wetting the coating using water, in order to let coagulate essentially the total proportion of resin of the paint coating and remove it from the substrate. The aqueous solution for treating substrates coated with lead-containing paint coatings, such as wood, metal, brickwork, clinkers resp. bricks and composition boards comprises: a solvent, which does not dissolve the paint coating and is compatible with water, a viscosity forming compound for preventing the falling off resp. dripping off the solution from surfaces arranged vertically or horizontally overhead, and an alkaline compound for reacting with pigment-containing portions of the paint coating in order to dissolve any crusted ingredients resp. compounds of lead present from the surface. In laboratory experiments, a wood substrate having a lead-containing paint coating is arranged in a horizontal plane, whereby the surface is oriented upwardly. After repeatedly applying the solution over a period of several hours, the paint coating begins to generate bubbles and detaches permanently from the substrate, whereby however the paint coating itself is not dissolved.

In summary, there is presently posed the problem to renovate resp. to remove and renovate, in particular to renovate resp. remove and renovate in-situ, paint and/or lacquer coatings charged with a hazardous material including e.g. a heavy metal, such as lead, because the conventional depainting methods, which are applied in-situ, essentially comprise working steps which are performed dry and which involve the generation of dust resp. flour.

It is an object underlying the present invention, to reduce the complexity and costs for protection from dust resp. for collecting dust, in particular dust comprising a hazardous material, when removing a coating comprising in particular the hazardous material, from a surface.

In all parts of the present document, the expression “resp.” is used as an abbreviation for the expression “respectively”, which is used herein in the meaning of “and/or”. The expression “dust” is to be understood as a collective denomination for fine solid particulate material resp. particles, which, once they have been dispersed, can float in gases, in particular in air, for a long time. A “priming coat layer” is to be understood as a layer resp. coating, which can be applied adhesively on a surface that is exposed resp. provided with an impregnation, which does not penetrate in the fine pores present at the surface, but covers the surface substantially in a two-dimensionally connected way, and which can provide an adhesion effect for the further build-up of coats of paint resp. lacquer. An “impregnation layer” is to be understood as a layer, which can been applied to an exposed surface, which can penetrate the surface, and which in any case protects the surface from detrimental influences resp. influences that may adversely affect the surface, from the environment, including e.g. moisture or fungal attacks.

It is, therefore, desirable to provide an improved method for removing a coating comprising a hazardous compound from a surface, which overcomes most, if not all of the preceding problems/disadvantages.

### BRIEF SUMMARY OF THE INVENTION

In order to solve the object, according to a first aspect of the invention, there is provided a method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a composition containing a heavy metal. The method comprises the following steps:
(i) pre-treating the coating to be removed resp. renovated, so that at least a portion of the coating is transformed into a moist resp. viscous state,
(ii) removing portions of the coating, which have been affected by the pre-treatment of step (i) at least in sub-areas of the portions, where the pre-treatment of step (i) has been applied, so that exposed parts of the surface are formed at least in these sub-areas.

According to the first aspect of the invention, the method further comprises:
(iii) applying, without performing an intermediate grinding beforehand, at least one priming coat layer at least in sub-areas of the exposed parts of the surface formed in step (ii).

In a first embodiment of the method according to the first aspect of the invention, step (i) may comprise applying a liquid agent for chemically stripping, macerating, etching resp. relieving onto at least portions of the coating to be removed, and step (ii) may affects the portions of the coating which have been effected by the liquid agent applied in step (i).

The method may comprise letting react a liquid agent resp. letting a liquid agent unfold its effect on the coating to be removed resp. renovated and comprising the hazardous material prior to relieving resp. removing of at least said portion of the coating from the surface.

After the relieving resp. removing of at least said portion of the coating from the surface, and without performing an intermediate grinding beforehand, a priming coat layer may be applied.

In a second embodiment of the method according to the first aspect of the invention, step (i) may comprise heating up of at least portions of the coating to be removed, and step (ii) may affects the portions of the coating which have been effected by the heating performed in step (i). The heating up may be performed using a heat torch resp. a hot air blowing device for producing a flow of heated air, for example a heat gun. The temperature of the heated air may be controlled to remain below a maximum threshold temperature selected so that a liberation of the hazardous material, which is contained in a gaseous composition expelled from the material of the coating to be removed resp. renovated, is essentially avoided. The maximum threshold temperature is 1100° F. (593° C.), and more preferably 750° F. (399° C.).

According to a second aspect of the invention, there is provided a method for removing resp. renovating a layer resp. coating, in particular a paint resp. lacquer coat, from a surface covered with the layer, wherein the coating to be removed comprises a hazardous material, for example a material containing a heavy metal, such as lead, an anorganic heavy metal compound or a compositions containing a heavy metal.

According to the second aspect of the invention, a working step, which produces dust, such as for example a grinding or a sanding, is performed at the earliest on, resp. in a portion of, the surface, which has been coated with at least one newly applied priming coat layer.

Before applying the new priming coat layer, at least portions of the coating comprising the hazardous material and having been relieved from the surface, may be removed from the surface without generating dust.

The removing of portions of the coating may pertain to portions of the coating which have been pre-treated for transforming into a moist resp. viscous state, for example by applying a liquid agent and/or by performing a heating treatment.

The method may further comprising a step, which is performed before applying the priming coat layer, of removing portions of the layer macerated, etched resp. relieved by the effect of a liquid agent.

A treatment step, which generates dust resp. flour, is performed only down to resp. within a working depth, which is less than the layer thickness (D) of the newly applied priming coat layer.

The treatment step, which generates dust, may comprise aspirating loose particles and particulate material formed by the removing of portions of the layer, in particular by using an aspiration device.

The priming coat layer may be applied without having performed any prior intermediate grinding beforehand.

According to a third aspect of the invention, there is provided a method for removing resp. renovating a layer resp. coating, in particular a paint resp. lacquer coat, from a surface covered with the coating, wherein the coating to be removed comprises a hazardous material, for example a material containing a heavy metal, such as lead, an anorganic heavy metal compound or a compositions containing a heavy metal.

According to the third aspect of the invention, at least a portion of the coating comprising the hazardous material is transformed into a moist resp. viscous state and is relieved resp. removed from the surface without generating dust and/or flour.

The transforming into a moist resp. viscous state may comprise applying a liquid agent resp. performing a heating treatment on the coating to be removed resp. renovated.

After the step of relieving resp. removing of at least said portion of the coating from the surface, without performing an intermediate grinding beforehand, a priming coat layer may be applied.

According to a fourth aspect of the invention, there is provided a method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a compositions containing a heavy metal.

According to the fourth aspect of the invention, remainders of the coating to be removed resp. renovated, which remain, after a step of relieving resp. removing loose resp. viscous portions of the coating from the surface without generating dust, in recesses, e.g. holes and pores, extending deeper into the otherwise substantially exposed surface, are covered resp. masked.

According to a fifth aspect of the invention, as an alternative or in addition to the fourth aspect, remainders of the coating to be removed resp. renovated, which remain attached to the surface after a step of relieving resp. removing loose resp. viscous portions of the coating from the surface without generating dust, are covered resp. masked.

In the method according to the third to fifth aspect of the invention, said covering resp. masking may be performed by applying an impregnating agent and/or a priming coat layer. The impregnating agent and/or the priming coat layer may be applied onto said otherwise substantially exposed portions of the surface.

The method according to the second to fifth aspect of the invention may further comprise applying a priming coat layer on a portion of the surface, and performing a treatment step, which generates dust, such as a grinding or sanding, at the earliest at least on portions of the surface, which have been covered resp. masked with at least one newly applied priming coat layer.

The treatment step, which generates dust resp. flour, is performed only down to resp. within a working depth, which is less than the layer thickness of the newly applied priming coat layer. Accordingly, possible remainders of coating to be removed resp. renovated, which comprise the hazardous material and which are located underneath the newly applied priming coat layer, remain covered resp. masked and hence untouched.

A treatment step, which generates dust, may comprise aspirating loose particles and particulate material formed by the removing of portions of the layer, in particular by using an aspiration device.

The removing resp. renovating of the coating may comprise wetting resp. moistening the coating using a liquid agent.

The step of applying the liquid agent may be selected from a group of treatment steps which comprises: brushing and spraying of the agent at least onto sub-areas of the layer and immersing of at least sub-areas of the surface covered by the layer to be removed resp. renovated into the liquid agent, or combinations thereof.

In the method according to the second to fifth aspect of the invention, the following steps may be performed prior to the application of the priming coat layer: (i) pre-treating the coating to be removed resp. renovated, so that at least a portion of the coating is transformed into a moist resp. viscous state, (ii) removing portions of the coating, which have been affected by the pre-treatment of step (i) and which are macerated, etched resp. relieved at least in sub-areas of the portions, where the pre-treatment of step (i) has been applied, so that exposed parts of the surface are formed at least in these sub-areas.

Prior to applying the priming coat layer without performing an intermediate grinding beforehand, an impregnating agent resp. an impregnating coat may be applied.

The method may further comprise, prior to the step of applying the priming coat layer, a step of cleaning of the surface using a liquid. The cleaning may comprise applying a film of liquid at on portions of the surface and subsequently removing, for example wiping off, the film.

The priming coat layer is preferably free of dichlore methane and free of n-methyl-pyrrolidone (NMP).

The method according to the first to fifth aspect of the invention may be performed in-situ on the surface.

According to a sixth aspect of the invention, there is provided a method for verification of the conformity, during work in progress on an object resp. article to be treated and comprising a hazardous material, with regulations on the limitation of the release of a hazardous material resp. regulations for the protection of the environment or of a person charged with the work in progress from a charge with the hazardous material, the method comprising:
performing a removing resp. renovating method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a compositions containing a heavy metal, according to any one of the first to fifth aspect of the invention as described above and at least one step, which is selected from a group comprising the following:
   (a) demounting resp. disassembling an object resp. article to be treated and transport to a treatment site, where protection equipment resp. protection precautions as required according to effective regulations are provided,
   (b) measurement of the charge of the environment, including the air resp. surfaces that are studdable with flour (resp. dust), with the hazardous material prior to the beginning of the execution of the removing resp. renovating method the coating in the surroundings of the demounting resp. demounting site, resp. in the surroundings of the treatment site,
   (c) measurement of the charge of the environment with the hazardous material during resp. after the execution of one of the steps according to the removing resp. renovating method resp. step (a), or
   (d) medical examination of at least one person who executes or assists to at least one method step of the removing resp. renovating method as to a charge with the hazardous material, or any combination of the steps (a) to (d).

The method for verification may comprise verifying that at least during one of the steps (b) and (c) at least one prescribed limiting value for the charge of the environment with the hazardous material is observed and/or verifying that during step (d) at least one prescribed limiting value for the charge of the person with the hazardous value is observed.

In the method for verification, all prescribed limiting value for the charge of the environment and/or all prescribed limiting values for the charge of the person may be observed.

The method for verification may comprise obtaining an obtaining a certificate of exemption, which exempts at least from one duty of the conformity to a regulation for the protection of the environment resp. a person charged with the work in progress, whence at least one, preferably all, of the verifications of the observation of prescribed limiting values has been provided.

In the method for verification and the methods for removing resp. renovating a layer resp. a coating comprising a hazardous material, from a surface, the hazardous material may be lead and a prescribed maximum limiting value of its concentration in air may be 0.3 mg/m3, preferably 0.1 mg/m3.

In the method for removing a layer resp. coating from a surface provided with the layer resp. coating, according to the first to fifth aspect of the invention, method steps generating dust are avoided during the removal of the layer. The layer may in particular be a lacquer resp. paint coating. The layer resp. coating to be removed may further comprise a hazardous material, in particular a heavy metal, such as lead, an inorganic heavy metal compound and/or a composition containing a heavy metal. The method for removing the layer resp. coating may be performed in-situ, i.e. in particular without dismounting resp. transporting to another working side resp. a work shop of at least portions of the surface.

Avoiding working steps generating dust during the removal of the layer resp. coating may be achieved in particular by providing that the step of removing of the layer comprises a step of wetting resp. humidifying the layer with a liquid agent, in particular an agent for chemically stripping, macerating, etching resp. relieving the layer. As a result of the wetting resp. humidifying the layer using a liquid agent, the material of the layer to be removed remains in a bound state due to the wetness resp. humidity, so that any generation of dust is avoided and the humidified layer can be removed from the surface without greater complexity and in particular without generating dust, can be collected and disposed off By wetting resp. humidifying the layer resp. coating with the liquid agent, the material of the layer resp. coating to be removed remains integral, wet and/or moistly, and remains bound without any generation of dust.

A working step which produces dust, such as in particular a grinding, is preferably performed only on resp. in a portion of the surface, which has been coated with at least one newly applied priming coat layer. The working step generating dust, such as in particular grinding, may be required in order to smoothen the surface resp. to prepare the surface for further treatment steps. As a result of limiting the dust producing working step to a portion of the surface, which has been coated with the newly applied priming coat layer, the generation of dust containing the material of the layer to be removed, which would otherwise occur, is avoided.

In particular, the dust producing working step, which is performed only on the newly applied priming coat layer, may be performed only within a depth, that is less than the layer thickness of the newly applied priming coat layer. The limitation of the working depth of the dust producing working step avoids safely and securely the generation of dust containing the material, the material which may possibly contain a hazardous material, of the layer which was initially to be removed from the surface.

The layer to be removed may in particular be a lacquer resp. paint coating. The layer to be removed may further comprise a hazardous material, in particular a heavy metal, such as lead, an inorganic heavy metal compound and/or a composition containing a heavy metal. In particular, the method for removing the layer can be performed in-situ, that is in particular without dismounting at least a portion of the surface resp. transporting it to another working site resp. to a work shop.

In the first embodiment of the first aspect of the invention, as a result of the effect of the liquid agent in step (ii), the material of the layer to be removed remains bound in a wet/humidified, gel-like and/or macerated state, in any case in a state in which no dust is generated. With respect to step (iii) it is emphasized, that this is executed without that a dust generating step, such as a grinding, is performed beforehand. As a result of applying the priming coat layer in step (iii) onto exposed parts of the surface, remainders of the layer to be removed, which are present in recesses that project a deeper into the surface, such as holes and/or pores, and which have not being reached resp. removed in step (ii), are covered resp. masked by the priming coat layer and are bound in this manner.

In the sub-areas generated in step (ii), in which exposed portions of the surface are generated, remainders of the layer resp. coating to be removed may remain on the surface, in particular in recesses resp. deep pores of the surface.

In step (i), the application of the liquid agent may be repeated several times, in particular as many times as is required to chemically strip, macerate, etch resp. relieve the layer to be removed over a substantial range of its thickness. As a result, the effect resp. the efficiencies of the liquid agent is improved.

The liquid agent may be a paint stripping agent for chemically stripping off the lacquer resp. paint coat. Advantageously, the liquid agent is an agent, which does not contain any hazardous or otherwise dangerous material, and/or for which during its application no elaborate personal protection measures are required, which go beyond the common usage of respiratory filters, protection goggles and common protection clothing and shoes. Preferably, paint stripping compounds which do not contain dichlore methane or n-methyl-2-pyrrolidon (NMP) are used. As paint stripping compounds, which are particularly inoffensive according to their classification by the GISBAU code resp. their declared composition, products of the brand “Meyer BA Uni” may be used.

GISBAU is the information system for hazardous materials of the professional association for construction projects (German: Berufsgenossenschaft BAU) established in Germany.

Advantageously, the liquid agent has a permission for being applied in the working area, in particular at a construction site, so that it can be used by the persons charged with the removal of the layer, without other persons in the working area, in particular at the construction site, being entangled. In particular, the liquid agent may have a declaration of hazardous materials having a GISBAU-code. At least in Germany, the GISBAU code is documented on a datasheet and may permit the usage in a working area, in particular at a construction site. In particular, the liquid agent may comprise an ecologically compatible resp. biologically degradable solving resp. dissolving agent.

The application of the liquid agent in step (i) may be chosen from a group of treatment steps comprising brushing and spraying onto at least portions of the layer to be removed and immersion of at least portions of the surface having thereon the layer to be removed into the liquid agent, or combinations of these steps. These treatment steps result in a homogenous application of the liquid agent.

In step (ii), the removing of portions of the layer may comprise a mechanical removing, in particular an abrasive removing, such as scraping and/or rasping. For example, the abrasive removing may comprise the use of a scraping device, such as a spatula or scraper. The abrasive removing can easily be performed such that the layer, which has been macerated, etched, relieved resp. chemically stripped by the liquid agent, is removed almost completely from the surface, without damaging the surface itself or appreciably ablating the surface.

Step (ii) may further comprise aspirating loose particles and particulate material formed by the removing of portions of the layer. To this end, an aspiration device may be used in particular.

Preferably, step (ii) is performed with a time-lag after step (i), whereby the time-lag is chosen so that the layer to be removed, by the effect of the liquid agent applied in step (ii), is dissolved, macerated, gel-like resp. moistly, in particular sufficiently resp. still dissolved, macerated, gel-like resp. moistly and has in particular not yet dried again. In this way, the formation of fine dust particles is avoided in step (ii) during the removing of portions of the layer, and portions of the removed layer, which have greater dimensions, e.g. crumbs, may be collected completely without further ado and put into a container.

Step (ii) may further comprise etching portions of the layer, which remain after a first attempt of removing, resp. collecting loose parts of the layer, which have been removed, in containers, which are sealable dust-proof. Preferably, the containers are also sealable emission-free. In that way, the remainders of the layer, which have been removed from the surface, can be disposed lead-safe and dust-free.

The method may further comprise, between the step (ii) and the step (iii), a step of cleaning the surface, which remains from step (ii), by using a liquid. The cleaning using the liquid results in a still more efficient removal of stripped-off parts resp. crumbs of the layer from the surface.

The cleaning may comprise applying a liquid film at least on portions of the surface remaining from step (ii) and a subsequent removing, in particular by wiping off the film. In particular, the cleaning may comprise a wiping off using a sponge resp. a sponge-like object soaked with the liquid, whereby the cleaning step can be performed in a single work step. The cleaning may comprise a wiping-off using a sponge soaked with the liquid, in particular water.

The liquid used in the cleaning step may be chosen from a group comprising: a neutralisation agent for neutralising an effect of the liquid agent used in step (i), a solvent for the lacquer resp. paint material contained in the layer to be removed, and water, or combinations thereof.

The method may comprise an intermediate grinding which is performed after step (iii) at least in sub-areas of the sub-areas provided with the priming coat layer in step (iii). The intermediate grinding prepares the primed surface even better for subsequent steps of treatment resp. coating. Since it is restricted to the sub-areas of the surface provided with the priming coat layer, the intermediate grinding does not generate any dust of the material of the layer that was present on the surface initially.

Preferably, the intermediate grinding is performed with simultaneous aspiration of dust resp. flour particles formed thereby. For wood surfaces, the aspiration may be performed in particular using dust filters of the dust class M (according to DIN EN 60335-2-69), which is prescribed for dust generating activities involving wood. The aspiration accompanying the intermediate grinding represents an additional protection against a possible liberation of the material of the layer, which initially covers the surface, into the air and/or the environment.

The intermediate grinding may be performed using small electric devices and/or by hand using grinding pads resp. grinding paper. In that way, the working depth of the intermediate grinding can be inspected resp. controlled particularly well, and can in particular be limited to the layer thickness of the priming coat layer.

As an impregnating agent and as a primer coat agent, products of the brand “Sigma Coatings” may be used, e.g. the products “SIGMALIFE Impregnant” as the impregnating agent, and “SIGMA AMAROL Triol Satin” or “SIGMA Holz-Iso-Grund” as the priming coat agent.

The intermediate grinding may be limited in particular with respect to its working depth to a maximum depth, which is less than the layer thickness of the priming coat layer applied in step (iii). In that way, it is ensured, that the remainders of the initial layer, which have been masked by the priming coat layer, remain bound on the surface.

The method according to the first to fifth aspect of the invention is advantageous in particular, when the layer to be removed contains a hazardous material, in particular a heavy metal, such as lead, an inorganic heavy metal compound and/or a composition containing a heavy metal. By the facts that the relieved portions of the layer containing the hazardous material remain bound as a result of the effect of the liquid agent and can, consequently, be removed from the surface without any generation of dust and be disposed off, and that remaining portions of the layer to be removed are masked by the priming coat layer and are therefore also bound, any uncontrolled release resp. liberation of the hazardous material e.g. into the environment, in particular by the generation of dust, is avoided. Consequently, it may in individual cases be considered to exempt from the provision of protection measures, which would otherwise be required according to applicable regulations when handling the hazardous material.

The surface, from which a layer resp. coating is to be removed, may be a metal surface or a wood surface. The method according to the first to fifth aspect of the invention is not limited by the type of the substrate material, to which the surface belongs.

An impregnation agent resp. an impregnation layer may be applied before the application of the priming coat layer. As a result, the surface, such as the metal or wood surface, is better protected from influences of the environment, such as weathering and fungal attacks, which may have become necessary as a consequence to the removal resp. renovation of the layer that was initially present on the surface.

The method according to the first to fifth aspect of the invention as described above features that before application of a priming coat layer (or an impregnation layer), any dry treatment step, which could generate dry loose particles, such as dust and/or crumps, of remainders of the layer resp. coating to be removed resp. renovated, in particular any grinding or sanding, is avoided. Rather, the material of the coating is transformed in a moist and/or viscous state so that as a consequence, a hazardous material comprised therein remains bound. This ensures that parts of the coating comprising the hazardous material are not liberated in an uncontrolled manner. As a result, there is a perspective that the compliance with possible regulations applicable for the handling of matter containing the hazardous material, in particular the provision of elaborate protection measures, may be refrained from.

A more detailed explanation of the invention is provided in the following detailed descriptions and appended claims taken in conjunction with the accompanying drawings.

### BRIEF DESCRIPTION OF THE DRAWINGS

Further, advantages embodiments, features and advantages of the invention follow from the following detailed description in relation with the appended drawings, in which:

FIG. 1 shows a vertical cross-section through a surface having disposed thereon a layer to be removed;

FIG. 2 shows a vertical cross-section through the surface of FIG. 1 during the application of the liquid agent for chemically stripping resp. etching;

FIG. 3 shows a vertical cross-section through the surface of FIG. 2 after the action of the liquid agent;

FIG. 4 shows a vertical cross-section through the surface of FIG. 3 during an abrasive removing of the layer;

FIG. 5 shows a vertical cross-section through the surface of FIG. 4 after the removing of the layer treated with the liquid agent;

FIG. 6 shows a vertical cross-section through the surface of FIG. 5 after applying an impregnating agent resp. an impregnating coat; and

FIG. 7 shows a vertical cross-section through the surface of FIG. 6 during the application of a priming coat layer.

### DETAILED DESCRIPTION OF THE INVENTION

In the following, embodiments of the method according to the first to fifth aspect of the invention are described by way of example for an application of removing lacquer resp. paint coatings from a surface, in particular a wood surface. The wood surface may be located in resp. belongs to a building, which is radically renovated. The wood surfaces may for example belong to wood-tiled walls or ceilings or to a plurality of wooden window frames, wherein a wooden window frame comprises a window frame fixedly anchored with a brick work and a movable window sash hinged therein and comprising a frame made of wood for framing a glass panel. The outer side of the window was exposed to weathering influences, so that the outer side surfaces of the window have to be renovated resp. removed.

Until in the 1980's, lacquers and paints have been used for wood surfaces as coatings, which comprise lead-containing hazardous materials. The methods which commonly in use today for removing the lacquer resp. paint coatings from wood surfaces (methods for depainting) are so-called dry methods, i.e. they comprise abrasive treatment steps for removing the coating, which are performed on the dry surface and/or the dry layers resp. coatings, such as e.g. a scraping or grinding. The development of dust is particularly strong, when grinding is performed. When scraping is performed, particles of greater or smaller size of remainders of the initial coating are generated, which crumble and thereby also develop dust.

Because of the containment of lead in the paint resp. lacquer coatings, these conventional methods for depainting are, in Germany, subject to the TRGS (German: technische Regel far Gefahrstoffe; technical regulation for hazardous materials) No. 505. This regulation prescribes among others, that it is not allowed to work without special protection measures when handling working materials furnished with lead-containing hazardous materials. The prescribed protection measures comprise an encapsulation of sources of dust, the provision of suction devices for suctioning the encapsulated core work areas, where the abrasive, dust-generating work is performed, the provision of sewer gates for entering and exiting the encapsulated core cell and the provision in the sewer gates of cleaning devices and rooms for changing clothes enabling changing the clothing of the persons performing the depainting work. The encapsulation in particular of window frames must be performed dust-safe to the outer and to the inner side.

The protection measures mentioned above represent a significant logistic, cost-wise additional effort for the work to be performed. This effort would not be required, if the colour resp. lacquer coatings to be removed would not contain a hazardous material. In the latter case, only the personal protective equipment for the persons performing the work would be required, and these could e.g. be prescribed by regulations decreed by the commercial inspectorate.

By applying the method according to the first to fifth aspect of the invention, the development of hazardous material containing dust can be avoided and the paint resp. lacquer layers, which have been relieved resp. removed from the surface according to the invention, can be completely catched and collected in dust-safe sealable containers and be disposed off.

In response to providing of an according verification, that the charge with the hazardous material (e.g. charge by heavy metals) of the environment due to the depainting works and the charge of persons in charge of the work do not exceed demonstratably certain, e.g. prescribed by official regulations, maximum upper limits, a certificate of exemption may be obtained, according to which the compliance with a above-mentioned, logistically and cost-wise elaborate regulations of the TRGS 505 is decreed. In that case, only the personal protection measures resp. safety equipment for the protection of the persons in charge with the work have to be supplied. This enables significant logistical and cost-wise savings.

According to a practically tested embodiment of the method according to first to fifth aspect of the invention, the depainting work on wood surfaces is performed in-situ. Firstly, as a particular embodiment of step (i) of the method according to the first to fifth aspect of the invention, a paint stripping compound is applied onto the lacquer resp. paint coating to be removed from a wood surface, in particular by spraying or brushing. As the paint stripping compound, which embodies the liquid agent according to the invention, a commercially available paint stripping compound is chosen, which is inoffensive for interior and/or exterior work in the sense of protecting persons and the environment, and which does not require any further personal safety equipment for the persons charged with the work when adequate aeration is provided. The selection of the paint stripping compound proceeds for example on the basis of the GISBAU code provided on the datasheet of the agent. Preferably, paint stripping compounds which do not contain dichlore methane or n-methyl-2-pyrrolidon (NMP) are used. As paint stripping compounds, which are particularly inoffensive according to their classification by the GISBAU code resp. their declared composition, products of the brand “Meyer BA Uni” may be used.

The application of the paint stripping compound can be repeated multiple times, as often as required until the lacquer resp. paint coatings are adequately stained resp. pickled and/or etched. Thereby, time resp. opportunity is given to the paint stripping compound to penetrate into the paint resp. lacquer coating and to unfold its etching and/or staining effect. The paint resp. lacquer coating transforms into a moist, gel-like layer, which can relatively easily be relieved from the surface, under the influence of the paint stripping compound.

Next, as a particular embodiment of the first embodiment of step (ii) of the method according to the first to fifth aspect of the invention, there is performed the removing of the layer treated by the paint stripping compound, the so-called paint removal. This is performed using a scraping device, which may in particular an electro-mechanically driven scraping tool, and/or using a hand tool, such as a spatula. The removed remainders of the paint resp. lacquer coating treated by the paint stripping compound and transformed in particular gel-like, can easily be let to drop onto the floor, be collected there and be put into a dust-safe sealable container. Optionally, an aspiration of material may be performed during the paint removal, e.g. using a mobile suction unit having a suction hose and a suction nozzle provided at a distal end of the suction hose. Thereby, the suction nozzle is guided in the vicinity of the working area of the scraping tool very close to the tool, such that smaller particles of the removed layer, which would otherwise drop onto the floor, are aspirated into the suction nozzle.

After the paint removal, the exposed surface is cleaned in a moist manner, i.e. using a liquid agent. To this end, the exposed surface is wiped using a sponge soaked with clear water. Depending on the prescriptions of the manufacturer of the paint stripping compound, the after-cleaning may, instead of using water, also be performed using other liquid agents, such as a liquid neutralisation agent for neutralising the etching effect of the paint stripping compound or a solvent for additionally releasing resp. washing-out of lacquer resp. paint remainders out of recesses in the wood surface, which have not been reached by the scraping device resp. the spatula. As a neutralisation agent resp. a solvent, agents are selected on the basis of the information in the datasheet resp. the GISBAU code, which declares the agent as inoffensive for persons resp. the environment.

After the moist cleaning, a drying step is performed. To this end, it suffices to wait for a predetermined drying period, until the after-cleaned surface is air dried. The drying process may be accelerated by blowing using an air flow, in particular a heated air flow. This may be performed e.g. using a blower device, which expells pre-heated air, such as a warm air blower device, in particular a blow-dryer resp. a blow-dryer-like device.

Thereafter, a further treatment of the depainted wood surfaces is performed, in this case without prior intermediate grinding, using an impregnating agent and a priming coat agent. An impregnating agent is applied without intermediate grinding onto the depainted wood surface, in particular by spraying resp. brushing. Thereafter, also without intermediate grinding, a priming coat agent is applied, and in this manner, there is applied as priming coat layer on the surface treated as described above.

The impregnating agent and/or the priming coat agent is selected on the basis of the information of the datasheet of the manufacturer resp. the GISBAU codes. Only those agents are selected, which are classified as an being inoffensive with respect to persons and the environment. As an impregnating agent and as a primer coat agent, products of the brand “Sigma Coatings” may be used, e.g. the products “SIGMALIFE Impregnant” as the impregnating agent, and “SIGMA AMAROL Triol Satin” or “SIGMA Holz-Iso-Grund” as the priming coat agent.

After the drying of the priming coat layer, an intermediate grinding may be performed for the first time. This is performed using small (electrical) devices resp. by hand using grinding pads. The intermediate grinding is performed substantially only down to a limited working depth, which is less than the layer thickness of the previously applied primer coat layer. The intermediate grinding serves to remove particularly noticeable resp. great elevations projecting out of the wood surface, which have been present before the beginning of the depainting work, which may have been created in the course of the depainting work, in particular during the paint removal, e.g. by the action of the scraping device. As an additional safety measure, the intermediate grinding is being performed with an adequate aspiration of dust, in particular using a dust filter of the dust class M (according to DIN EN 60335-2-69).

The further build-up of the paint resp. lacquer layer system is applied onto the priming coat layer resp. onto the intermediately grinded priming coat layer. For example, in accordance with the products of the brand “Sigma Coatings”, the product “SIGMA AMAROL Triol Satin” may be applied as an intermediate and/or final coating, where necessary also multiple times, and the products “SIGMA AMAROL Triol gloss” or “SIGMA CONTOUR gloss”, which lead to a glossy appearance, may be applied as the final coating

In order to verify before the official agency resp. the commercial inspectorate, which would the supervise the compliance with the regulations of the TRGS 505, that the charge of the environment and/or the persons charge with the performing of the activities is inoffensive resp. may be neglected for being within predetermined maximum limiting values or not being present at all, so that the application of the measures prescribed according to the TRGS 505 may be decreed with, the actions described in the following may be undertaken.

A portion of the surface, from which the paint resp. lacquer coating is to be removed, e.g. a window frame made of wood and being provided with a paint coating to be renovated, is dismounted under the application of customary dust protection measures, packed (and preferably sealed dust-proof) and transported to a working site resp. a work shop, which has the safety equipment and/or personal safety equipments as required, e.g. according to the TRGS 505. The dismounting and the packing is accompanied by measurements of the hazardous material from the very beginning, whereby the measurements are performed by an independent organisation recommended by the official agency or the commercial inspectorate.

After the working site resp. the work shop has been provided with the required protection equipments resp. personal protection equipments, the dismounted part of the surface, e.g. the dismounted window frame, is established in the work shop resp. at the working site. There, for all safety, an aspiration unit resp. a dust aspiration device is provided. As personal safety equipment, there may be provided safety clothing, dust respirators provided with particle filters, in particular of the type P2, safety gloves in particular nitrile gloves, protective goggles and protective shoes.

In the surroundings of the working site resp. the work shop, the charge with the hazardous materials is measured prior to the start of the activities and during the performing of the activities, in particular by said independent organisation.

The persons charged with the work are examined medically. In particular, the screening examinations for detecting a possible charge with the hazardous material prescribed according to the TRGS 505 may be performed. This holds for the activities at the working site resp. work shop and also for the activities and work performed in-situ, i.e. at the site, where the wood surfaces to been depainted are present, in particular at the construction site.

In the course of the treatment steps according to the invention as described above, which are performed in-situ, measurements of the hazardous material may be performed for verifying the inoffensiveness of the method according to the invention, in particular by one or more independent recognized organisations. These measurements may be performed in particular before the beginning of the depainting work resp. the chemically stripping step, during the chemically stripping step (step (i)), during the paint removal and the collection of the remainders of the layer in the dust-safe sealable containers, during the after-cleaning, and/or during the intermediate grinding on the newly applied priming coat layer.

In the following, a method according to the first embodiment of the first aspect of the invention as described above is described with reference to FIGS. 1 to 7.

Initially, a paint resp. lacquer layer resp. coating 4 is present on the surface 2. As indicated in FIG. 1, the wood surface 2 has a predetermined surface roughness resulting in particular from the texture of the wood and/or from the treatment steps that have been performed during the manufacture of the surface 2.

As illustrated in FIG. 2, a chemically stripping compound resp. a film of a chemically stripping agent 6 is applied on the paint resp. lacquer coating 4, for example using a paint brush 8. The liquid chemically stripping compound penetrates into the paint resp. lacquer coating 4, in particular by capillary action, diffusion and dissolving processes. That is, the chemically stripping compound 6 penetrates the paint resp. lacquer coating 4 as is shown in FIG. 2 by the downwardly directed arrows shown below the film 6 of the chemically stripping compound.

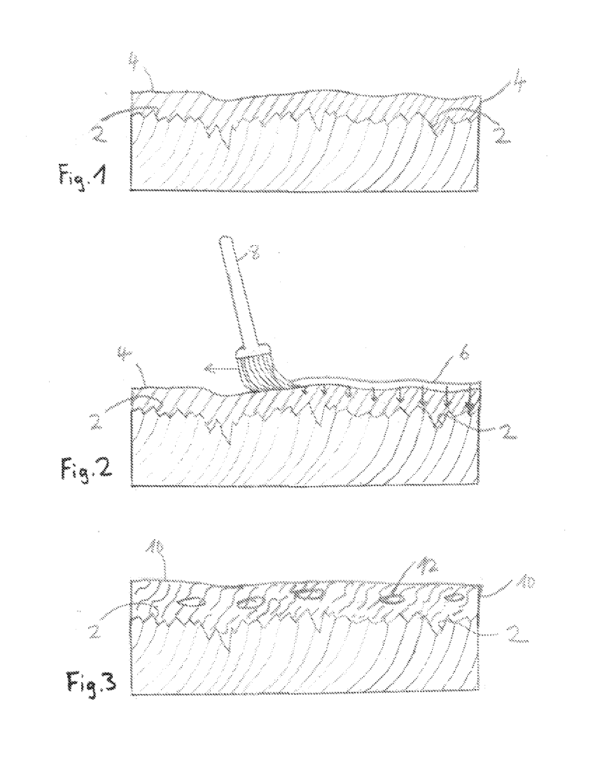

As illustrated in FIG. 3, the chemically stripping compound 6, which has penetrated in the paint resp. lacquer coating 4, causes an etching resp. chemically stripping of the paint resp. lacquer coating 4. Thereby, the coating 10 treated with the chemically stripping compound 6 swells, gas bubbles 12 may form therein, and the coating transforms such that a gel-like, in particular moist, consistency is formed. Thereby, the layer remains substantially bound and a generation of dust does not happen resp. is, in effect completely, avoided.

After the action of the chemically stripping compound 6 the macerated layer 10 is scraped resp. removed from the surface 2 using a spatula 14, in a very gently abrasive, in particular scraping, manner. In the thus treated area 16 of the surface, the surface roughness of the surface may change under the action of the scrapping device 14, and may in particular be flattened, as is illustrated in FIG. 4 in the left portion at the reference numeral 16.

As illustrated in FIG. 5, after the abrasive removing of the chemically stripped layer 10, there remains a surface, on which substantially exposed portions 22 of the surface 2 are present and on which there remain only insular remainders 20 of the layer. Remainders of the layer may be localized in particular in recesses 18, which have not been reached by the scraping device 14.

The substantially exposed surface 22 is optionally after-cleaned using a liquid, for example by wiping with a sponge soaked with water and thereafter dried (not shown).

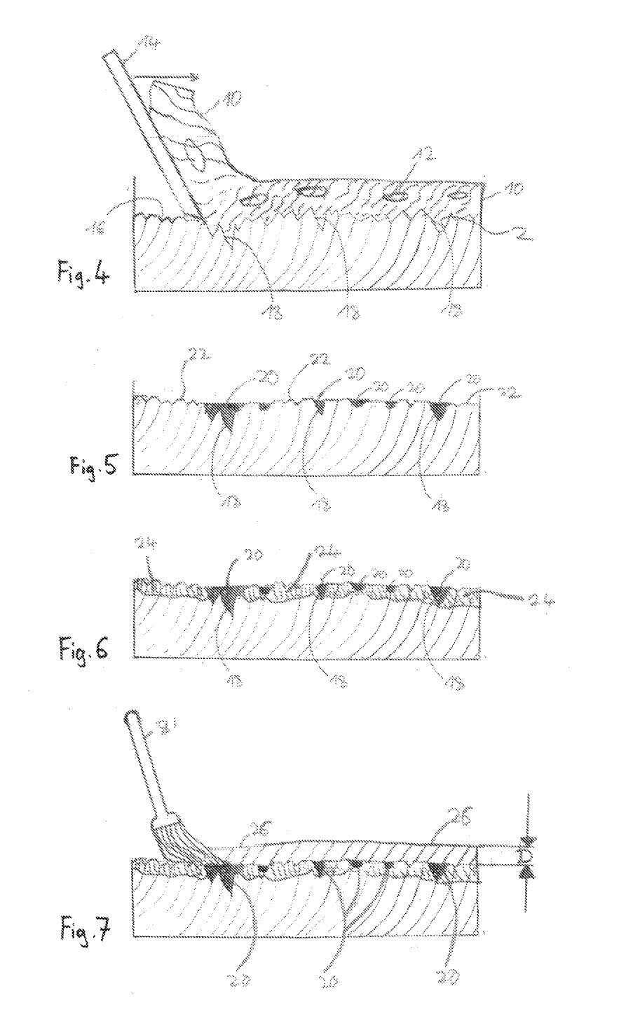

An impregnating agent is applied onto the substantially exposed, and after the optional after-cleaning also dried surface 22, in particular by spraying or brushing. The impregnating agent penetrates the exposed surface 22, i.e. it penetrates the latter and forms at resp. under the surface, in particular in the exposed portion of the exposed surface 22, an impregnated layer 24, as shown in FIG. 6.

Then, a priming coat layer is applied, for example by spraying or, as shown in FIG. 7, by brushing using a paint brush 8′. The priming coat agent, which is in particular liquid, has a relatively high viscosity resp. a high concentration of pigments, so that it does substantially not penetrate the rough resp. porous wood surface 22 resp. 2, but settles on the surface 2 resp. 22. Time resp. opportunity for drying is given to the applied film of priming coat agent, so that a priming coat layer 26 is formed on the surface 2, 22. The applied priming coat layer 26 has a layer thickness D, as shown in FIG. 7.

Optionally, an intermediate grinding may be performed on the priming coat layer 26 (not shown). The intermediate grinding is performed only within the layer thickness D of the priming coat layer 26, i.e. the working depth is less than the layer thickness D. Thus, it is ensured, that remainders 20 of the initial coating, which have been covered, so-called “masked” by the priming coat layer 26, are not exposed, but remain covered by the priming coat layer 26.

Any paint resp. lacquer built-up of coats that is compatible with the priming coat layer 26 can then be applied onto the priming coat layer 26.

The embodiment of the method according to the invention described above has been described for the removal of a paint resp. lacquer coating from a wood surface. However, the method is not limited to this, but may also be applied for removing layers resp. coatings from other types of surfaces, in particular from metal surfaces.

Novel and advantageous features of the method according to the first to fifth aspect of the invention are, that for the removal of the layer resp. coating from the surface processing steps, which would generate dust, are avoided and are performed at the earliest after the application of a priming coat layer, which masks eventually remaining remainders of the coating. A further novel and advantageous feature is, that a liquid agent for etching, relieving resp. chemically stripping the coating is applied (step (i)), whereby small loose particles and dust remain bound resp. their liberation is disabled resp. avoided. A further novel and advantageous feature is, that the removing of the layer in step (ii) is performed, when the coating is in a moist, for example gel-like state as a result of the action of the liquid agent. A still further novel and advantageous feature is, that no abrasive treatment step, in particular no grinding, which would generate dust/flour is performed on the surface, which is exposed in step (ii). Instead, an impregnation is optionally applied and in any case a priming coat layer is applied in step (iii). As a result, possible remainders of the layer, which may have remained after having performed step (ii), e.g. in recesses of the surface or greater troughs and/or basins of the surface which is not perfectly planar, which facilitate the presence of remainders from step (ii) of the initially present coating to be removed, are masked resp. covered and thus bound. A still further novel and advantageous feature is that a first intermediate grinding, resp. any grinding, is performed at the earliest on the priming coat layer that has been newly applied in step (iii).

### EXAMPLE

The method according to the first to fifth aspect of the invention has been made known among skilled persons and official agencies by the inventor under the name RISAN® method. The RISAN® method has been applied and tested e.g. at several sites of renovation work performed on wooden window frames and window saches of historical buildings in Munich (Germany), including the residential palace (“Residenz Königsbau”) and the museum of ethnology (“Völkerkundemuseum”), of a primary school at St.Annastraβe that is regularly visited by children (“Grundschule St. Annastraβe”), and of a construction site comprising a building to be renovated at Zieblandstraβe (“Baustelle Zieblandstraβe”). In all cases, the method was applied under close supervision, testing and performing measurements of the concentration of lead in the air and of the charge of workers in charge of the paint renovation work, by the professional association for construction projects involving wood (German: Holz Berufsgenossenschaft, abbreviated BG Holz) established in Germany.

Measurements of lead, of other metals and the total dust fraction in the air were performed in the working areas at the afore-mentioned renovation sites, while paint coat on the window frames were being chemically stripped. These measurements yielded the concentrations of (heavy) metals [mg] in air [m3] at the different sites as stated below.

At “Residenz Königsbau” and at “Völkerkundemuseum”, workers involved in the depainting work were medically screened, to measure the charge of lead, as determined by the concentration [μg/l] of lead [μg] in samples of blood [1].

At “Zieblandstraβe”, samples were taken of the material of the coating to be removed and the contents of lead and other metals were measured for these samples.

At “Residenz Königsbau”:
for lead at maximum 0.036 mg/m3 (corresponding to 1.3 mass %), and further samples showing less than 0.0024 mg/m3 and less than 0.0012 mg/m3, which all comply with the maximum permissible value of 0.1 mg/m3,
for chrome at maximum less than 0.0012 mg/m3 (corresponding to 0.16 mass %), which complies with the maximum permissible value of 2 mg/m3,
for cadmium at maximum less than 0.0012 mg/m3 (corresponding to 0.010 mass %) (no maximum permissible value prescribed), and
for cobalt at maximum less than 0.0012 mg/m3 (corresponding to 0.010 mass %) (no maximum permissible value prescribed).

Medical screening of three workers yielded the following lead concentrations in the blood, measured after the depainting work was finished:
for worker 1: 103 μg/l,
for worker 2: 183 μg/l, and
for worker 3: 241 μg/l.

The medical screening results for all three workers complies with the prescribed maximum permissible limit of 400 μg/l.

At “Völkerkundemuseum”

A worker involved in the depainting work in a pilot test study yielded lead concentrations in the blood, as measured before and after the depainting work as follows:
before beginning the depainting: 260 μg/l, and
after the depainting: 218 μg/l,
   which complies well with the prescribed maximum permissible limit of 400 μg/l.

The maximum permissible limit of 400 μg/l for the concentration of lead in blood prescribed in Germany is well below that of NIOSH in the United States, which prescribes a value of not more than 0.060 mg lead per 100 gram of blood (approx. 600 μg/l).

At “Grundschule St. Annastraβe”, as monitored during the total length of a work shift:
for lead at maximum 0.001 mg/m3 (corresponding to 0.48 mass %), which complies with the maximum permissible value of 0.1 mg/m3, and
for a total fraction of dust/aerosols 1.28 mg/m3 (corresponding to 0.71 mass %), which complies with the maximum permissible value of 10 mg/m3.

Also the liberation of solvents from the paint stripping compound into the air was measured and monitored during the total length of a work shift, with the following results:

| | Measured | prescribed maximum value |
|---|---|---|
| | [mg/m3] | [mg/m3] |
| dimethyl adipate | <0.4 | 1.0 |
| dimethyl glutarate | <0.4 | 33.0 |
| dimethyl succinate | <8.0 | 35.0 |
| 2-(2-ethoxy ethoxy) ethanol | <0.4 | 33.0 |
| hydro carbon composition | 42.0 | 1,000 |
| (2-methoxy methyl ethoxy) | <4.2 | 310 |
| propanol | | |

At “Zieblandstraβe”:
for lead at maximum 0.0051 mg/m3 (working site no. 2), and a further sample showing less than 0.0043 mg/m3 (working site no. 1), which all comply with the maximum permissible value of 0.1 mg/m3, and
for a total fraction of dust/aerosols at 2.45 mg/m3 (working site no. 2) and a further sample showing less than 0.52 mg/m3 (working site no. 1) (corresponding to 0.71 mass %), which complies with the maximum permissible value of 10 mg/m3.

A sample of material of the coating to be removed showed a concentration of metal [mg] per mass of the material [kg] as follows: lead 19.000 mg/kg, cobalt <300 mg/kg and chrome <50 mg/kg.

All features disclosed in the application documents are claimed as a being relevant to the invention, insofar as they are, individually or in combination, novel with respect to the state of the art.

### LIST OF REFERENCE NUMERALS

2 surface
4 layer resp. coating (e.g. of paint resp. lacquer)
6 paint stripping compound resp. film of paint stripping compound
8, 8′ application device (e.g. paint brush)
10 layer macerated using a chemically stripping compound
12 gas bubble
14 scraping device (e.g. spatula)
16 scraped surface
18 recess
20 remainder of layer
22 exposed surface
24 impregnated layer
26 priming coat layer
D layer thickness

The following is a detailed description and explanation of the preferred embodiments of the invention and best modes for practicing the invention.

Although embodiments of the invention have been shown and described, it is to be understood that various modifications, substitutions, and rearrangements of parts, components, and/or process (method) steps, as well as other uses, shapes, construction, and design of the method for removing a coating comprising a hazardous compound from a surface can be made by those skilled in the art without departing from the novel spirit and scope of this invention.

What is claimed is:

1. Method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a compositions containing a heavy metal, the method comprising the following steps:
(i) pre-treating the coating to be removed resp. renovated, so that at least a portion of the coating is transformed into a moist resp. viscous state;
(ii) removing portions of the coating, which have been affected by the pre-treatment of step (i) at least in sub-areas of the portions, where the pre-treatment of step (i) has been applied, so that exposed parts of the surface are formed at least in these sub-areas; and
(iii) applying, without performing an intermediate grinding beforehand, at least one priming coat layer at least in sub-areas of the exposed parts of the surface formed in step (ii).

2. Method according to claim 1, characterized in that:
step (i) comprises applying a liquid agent for chemically stripping, macerating, etching resp. relieving onto at least portions of the coating to be removed, and
step (ii) affects the portions of the coating which have been effected by the liquid agent applied in step (i).

3. Method according to claim 1, characterized in that:
step (i) comprises heating up of at least portions of the coating to be removed, and
step (ii) affects the portions of the coating which have been effected by the heating performed in step (i).

4. Method according to claim 3, characterized in that:
the temperature of the heated air is controlled to remain below a maximum threshold temperature, preferably 1100° F. (593° C.) and more preferably 750° F. (399° C.), selected so that a liberation of the hazardous material, which is contained in a gaseous composition expelled from the material of the coating to be removed resp. renovated, is essentially avoided.

5. Method for removing resp. renovating a layer resp. coating, in particular a paint resp. lacquer coat, from a surface covered with the layer, wherein the coating to be removed comprises a hazardous material, for example a material containing a heavy metal, such as lead, an inorganic heavy metal compound or a compositions containing a heavy metal, wherein a working step, which produces dust, such as for example a grinding or sanding, is performed at the earliest on, resp. in a portion of, the surface, which has been coated with at least one newly applied priming coat layer.

6. Method according to claim 5, characterized in that before applying the new priming coat layer, at least portions of the coating comprising the hazardous material are removed from the surface without generating dust.

7. Method according to claim 6, characterized in that the removing of portions of the coating pertains to portions of the coating which have been pre-treated for transforming into a moist resp. viscous state, for example by applying a liquid agent or by performing a heating treatment.

8. Method according to claim 5, characterized in that the treatment step, which generates dust resp. flour, is performed only down to resp. within a working depth, which is less than the layer thickness (D) of the newly applied priming coat layer.

9. Method according to claim 8, characterized in that a treatment step, which generates dust, comprises aspirating loose particles and particulate material formed by the removing of portions of the layer, in particular by using an aspiration device.

10. Method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a compositions containing a heavy metal, wherein at least a portion of the coating comprising the hazardous material is transformed into a moist resp. viscous state and is relieved resp. removed from the surface without generating dust and/or flour.

11. Method according to claim 10, characterized in that the transforming into a moist resp. viscous state comprises applying a liquid agent resp. performing a heating treatment on the coating to be removed resp. renovated.

12. Method according to claim 10, characterized in that after the step of relieving resp. removing of at least said portion of the coating from the surface, without performing an intermediate grinding beforehand, a priming coat layer is applied.

13. Method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a compositions containing a heavy metal, wherein remainders of the coating to be removed resp. renovated, which remain, after a step of relieving resp. removing loose resp. viscous portions of the coating from the surface without generating dust, in recesses, e.g. holes and pores, extending deeper into the otherwise substantially exposed surface, are covered resp. masked, and/or wherein remainders of the coating to be removed resp. renovated, which remain attached to the surface after a step of relieving resp. removing loose resp. viscous portions of the coating from the surface without generating dust, are covered resp. masked.

14. Method according to claim 13, characterized in that said covering resp. masking is performed by applying an impregnating agent and/or a priming coat layer.

15. Method according to claim 13, characterized in that a treatment step, which generates dust, such as for example a grinding or sanding, is performed at the earliest at least on portions of the surface, which have been covered resp. masked with at least one newly applied priming coat layer.

16. Method according to claim 15, characterized in that the treatment step, which generates dust resp. flour, is performed only down to resp. within a working depth, which is less than the layer thickness (D) of the newly applied priming coat layer.

17. Method according to claim 16, characterized in that the treatment step, which generates dust, comprises aspirating loose particles and particulate material formed by the removing of portions of the layer, in particular by using an aspiration device.

18. Method according to any one of the preceding claims, characterized in that it is performed in-situ.

19. Method according to any one of the claims 1 to 9, 12 and 14 to 18, wherein the priming coat layer is free of dichlore methane and free of n-methyl-pyrrolidone (NMP).

20. Method for verification of the conformity, during work in progress on an object resp. article to be treated and comprising a hazardous material, with regulations on the limitation of the release of a hazardous material resp. regulations for the protection of the environment or of a person charged with the work in progress from a charge with the hazardous material, the method comprising:
performing a removing resp. renovating method for removing resp. renovating a layer resp. coating, in particular a paint resp. lacquer coat, from a surface covered with the coating, wherein the coating to be removed comprises a hazardous material, for example a material containing a heavy metal, such as lead, an inorganic heavy metal compound or a compositions containing a heavy metal, and at least one step, which is selected from a group comprising the following:
(a) demounting resp. disassembling an object resp. article to be treated and transport to a treatment site, where protection equipment resp. protection precautions as required according to effective regulations are provided,
(b) measurement of the charge of the environment, including the air resp. surfaces that are studdable with flour (resp. dust), with the hazardous material prior to the beginning of the execution of the removing resp. renovating method in the surroundings of the demounting resp. demounting site, resp. in the surroundings of the treatment site,
(c) measurement of the charge of the environment with the hazardous material during resp. after the execution of one of the steps according to the removing resp. renovating method resp. step (a), or
(d) medical examination of at least one person who executes or assists to at least one method step of the removing resp. renovating method as to a charge with the hazardous material, or any combination of the steps (a) to (d).

21. Method according to claim 20, characterized by comprising:
verifying that at least during one of the steps (b) and (c) at least one, and preferably all, prescribed limiting value for the charge of the environment with the hazardous material is observed and/or
verifying that during step (d) at least one, and preferably all, prescribed limiting value for the charge of the person with the hazardous value is observed.

22. Method according to claim 21, characterized by comprising:
obtaining an obtaining a certificate of exemption, which exempts at least from one duty of the conformity to a regulation for the protection of the environment resp. a person charged with the work in progress, whence at least one, preferably all, of the verifications of the observation of prescribed limiting values has been provided.
